SEQUENCE LISTING (4 sequences)

SEQ ID NO: 1 - DNA, 36 bp, Artificial Sequence
  Description of artificial sequencePrimer
cgcggatcctgatgcgtgcactacgcaaaggccagg

SEQ ID NO: 2 - DNA, 36 bp, Artificial Sequence
  Description of artificial sequencePrimer
ccggaattcagtcagcgggccatggagcttgagccc

SEQ ID NO: 3 - DNA, 68 bp, Artificial Sequence
  Description of artificial sequencePrimer
tatatagcatgcaagaaggagatatacatatgccccgccccaagctcaagtccgatgacgaggtactc

SEQ ID NO: 4 - DNA, 44 bp, Artificial Sequence
  Description of artificial sequencePrimer
tttatagtcgaccagggactctgcacacctccgtttacgcatgt

(19) United States
(12) Patent Application Publication
(10) Pub. No.: US 2004/0209270 A1
(43) Pub. Date: Oct. 21, 2004

(54) Method for identifying macrocyclic polyketides

(76) Inventors: Gunther Eberz, Odenthal (DE); Volker Mohrle, Koln (DE)

Correspondence Address: BAYER CROPSCIENCE LP, Patent Department, 100 BAYER ROAD, PITTSBURGH, PA, 15205-9741, US

(21) Appl. No.: 10/486,187

(22) PCT Filed: Jul. 25, 2002
(86) PCT No.: PCT/EP02/08282

(30) Foreign Application Priority Data
Aug. 7, 2001 (DE) 101 38 766.0

(51) Int. Cl. C12Q 1/68; C12Q 1/66; C12Q 1/18

(52) U.S. Cl. 435/6; 435/32; 435/8

(57) ABSTRACT

The present invention relates to a method for identifying macrocylic polyketides by a biosensor system and to components of this biosensor system.

The present invention relates to a method for identifying macrocylic polyketides by a biosensor system and to components of this biosensor system.

Macrocylic polyketides are one of the pharmacologically most important natural product classes, since they are distinguished by a wide range of actions. They have a polyketide backbone with more than 10 atoms in a ring whose characteristic feature is at least one lactone or/and lactam bond. The macrolides, i.e. macrocylic polyketides with a lactone ring, include, for example, the antimicrobially active and clinically used 14- and 16-membered lactones carbomycin, erythromycin, leucomycin and oleandomycin. They inhibit protein biosynthesis by binding to the 50S subunit of the 70S prokaryotic ribosomes. In addition, there are the “non-classical” macrolides whose rings are comparable in size and which have no sugar building blocks, but are nevertheless biologically active, such as, for example, soraphen, albomycin or the anti-fungally and cytostatically active epothilone. Epothilones inhibit a wide range of tumour cell lines. Polyene macrolides contain 20- to 38-membered lactone rings and are frequently glycosylated with an amino sugar. They are anti-fungally active and are partly used clinically for fungal and yeast infections (e.g. nystatin). The spiro-macrolides include avermectins and milbemycins which have a large market in the control of endo- and ectoparasites in animals and as insecticides in crop protection. The pleco-macrolides have a folded side chain, in addition to the lactone ring as characteristic feature. These macrolides are highly active ATPase inhibitors such as, for example, concanamycin A. Macrodiolides (e.g. elaiophyllin) and macrotetrolides (e.g. nonactin) are composed in a characteristic manner of repetitive subunits. Examples of macrocyclic polyketides which have both a lactone and a lactam bond are the structurally similar immunomodulators rapamycin and FK-506. Rifamycin is likewise a macrocyclic polyketide in which a lactam bond rather than a lactone bond is present. Rifampicin is a semi-synthetic derivative of rifamycin with better pharmacological properties and a wider range of actions.

According to the current state of the art, it is not possible to detect macrocyclic polyketides in biological screening methods, for example of natural product extracts, on the basis of their characteristic structural property. Rather, the screening assay used in each case depends on the said substances being noticed due to the biological activity screened for in each case. Macrocyclic polyketides having a different or different actions than those screened for in the assay are not detected by this method. Owing to the extraordinary potential of biological efficacies of macrocyclic polyketides, however, it is desirable to selectively detect this class of substances independently of their action.

One possibility of detecting analytes is the use of biosensors. There exist various genetically constructed, biological components of a biosensor which indicate selectively cellular effects such as heat-shock activation or carcinogens, notwithstanding the structure of a chemical substance. A number of other biological biosensor components may, primarily in the presence of particular metal salts or particular cellular metabolites, develop measurable signals (Daunert et al., 2000). Such assay systems may have a high detection sensitivity, as has been shown, for example, for biosensors for detecting substances inhibiting nitrification (Ludwig et al., 1999) or chromate salts (Peitzsch et al., 1998). A biosensor system for the aromatic polyketide tetracycline has also been described (Kurittu et al., 2000). A sensor for detecting the pharmacologically attractive natural product class of macrocyclic polyketides has hitherto not been described.

The present invention relates to a method for identifying macrocyclic polyketides, in which method macrocyclic polyketides or a mixture containing macrocyclic polyketides are subjected to a biosensor system.

The method according to the invention provides, with application of a biosensor system the possibility of focusing a screening on the attractive natural product class of macrocyclic polyketides. Since the biological activity of a macrocyclic polyketide may be relevant to very different areas, such as, for example, to the field of health, of agriculture or of environmental protection, this focused process may considerably reduce the work needed for detecting novel biologically interesting macrocyclic polyketides.

The term “biosensor system”, as used herein, means in particular a system based on a genetically engineered, cellular biosensor. The biosensor comprises a biological component, a microbial detection system which is suitable for specifically detecting a macrocyclic polyketide. Detection of the macrocyclic polyketide is indicated by simulation of biochemical information. This biochemical information may be measured and quantified using a suitable technical component.

The biological component of the biosensor system for macrocyclic polyketides is preferably a cellular reporter-gene assay system. Such a cellular reporter-gene assay system particularly preferably comprises a reporter gene whose transcription is under the control of a promoter region which is regulated as a function of a macrocyclic polyketide.

Reporter genes which may be used are various genes such as, for example, genes coding for chloramphenicol acetyltransferase, beta-galactosidase, a bacterial or firefly luciferase or green fluorescent protein (GFP). Such reporter genes have proved useful in the development of biosensors (Daunert et al., 2000). Preference is given to using as reporter gene a gene coding for luciferase.

The biochemical information generated by the biological component may be recorded with the aid of imaging methods such as, for example, photographic methods, video imaging or else manual drawing. The biochemical information generated by the biological component may also be converted into a physically quantifiable signal by using a signal transducer as technical component. The said signal may be, for example, an electrical signal accessible to electronic amplification. Examples of transducers which may be utilized are an amperometric or potentiometric electrode, an optrode, a piezocrystal and a thermistor (Puhler et al., 2000).

The promoter used for controlling transcription of a reporter gene as a function of a macrocyclic polyketide is preferably the promoter region of the E. coli mph(A) gene. Regulation of the promoter region requires the MphR(A) protein which is preferably provided by overexpressing the corresponding gene from E. coli.

Accordingly, the biological part of the biosensor system is preferably based on components of an erythromycin-resistance gene operon from E. coli (Noguchi et al., 2000). According to the published model of this mph(A) operon, three gene products are encoded: Mph(A), Mrx and MphR(A). Here, expression of this operon is negatively regulated at the level of transcription, due to binding of MphR(A) to the promoter region of the mph(A) gene, the first gene of the said operon. In the absence of erythromycin, MphR(A) binds to the promoter region of the mph(A) operon and represses expression of the latter. The presence of erythromycin stops MphR(A) from binding to this promoter region, thus releasing expression of the operon (FIG. 1).

Both prokaryotic and eukaryotic cells may be used as host cells which are another biological component of the biosensor system for the method of the invention. Preference is given to using bacteria, particularly preferably E. coli, a gram-negative microorganism.

In order to prepare a very particularly preferred embodiment of a biosensor system for the method of the invention, the following components are prepared and combined: (i) a luciferase reporter-gene plasmid whose expression is under the control of the mph(A)-promoter region, (ii) an E. coli cell which overexpresses the mphR(A) gene and, at the same time, carries the mph(A) operon or parts thereof.

The procedure of preparing this very preferred embodiment is described in more detail below.

The luciferase reporter-gene plasmid mentioned above is prepared, for example, by amplifying, using PCR, the promoter region-encoding and MphR(A)-binding DNA sequence of the mph(A) operon of plasmid pTZ3509 (Noguchi et al., 2000) (cf. Example 1) and cloning this sequence into the promoter-screening vector pUCD615 (Rogowsky et al., 1987). This transcription fusion (operon fusion) vector encodes the promoter-less structural genes for Vibrio fischeri luciferase (lux genes). The promoter-carrying piece of DNA is cloned into pUCD615 in such a way that the mph(A)-promoter region controls transcription of the lux genes. The mph(A) operon which provides resistance to erythromycin is transferred by recloning an mph(A) operon-encoding DNA fragment from plasmid pTZ3509 into the transposon suicide vector pBSL180 (Alexeyev and Shokolenko, 1995) (cf. Example 2). The MphR(A) protein is overexpressed by obtaining the mphR(A) gene from plasmid pTZ3509 by PCR amplification and cloning it into the pACYC184 vector (Chang and Cohen, 1978) (cf Example 3).

An example of the E. coli cell used is SM101 (Vuorio and Vaara, 1992). This strain is an lpxA2 mutant which is defective in UDP-N-acetylglucosamine-acyltransferase activity. This enzyme is required for the first step of lipidA biosynthesis of the outer cell membrane. The lpxA2 mutation causes a drastic increase in sensitivity to hydrophobic antibiotics such as erythromycin or rifampin.

The luciferase reporter-gene plasmid whose expression is under the control of the mph(A)-promoter region, the mphR(A)-carrying plasmid and the mph(A) operon-transferring suicide plasmid are transferred into the bacterial strain E. coli SM101 by standard methods of molecular genetics.

The host cell used may also be an E. coli strain carrying a tolC mutation. A functional tolC gene product is required for an effective AcrAB-efflux system and for expressing the Mar phenotype (Fralick, 1996). It may play a part in secreting noxious hydrophobic substances. It is, of course, also easily possible to use as host organism for the formation of the biosensor system of the invention an E. coli strain which carries both an lpxA2 and a tolC mutation.

The procedure presented by way of example may be used to prepare a biosensor system which has the property of expression of the luciferase gene and thus luminescence being stimulated by 12- to 16-membered macrocyclic polyketides. The biosensor system of the invention indicates, for example, the 14-membered natural macrolidic protein synthesis inhibitors erythromycin, clarithromycin, oleandomycin, picromycin and narbomycin. The luminescence of the biosensor system of the invention is also stimulated by the 12-membered macrolide methymycin which is anti-bacterially active against Gram-positive bacteria. In addition, the luminescence of the biosensor system of the invention is likewise stimulated by a 15-membered, semi-synthetic macrolide, azithromycin. The biosensor system also indicates the macrocyclic lactam-polyketide rifampicin, a semi-synthetic rifamycin derivative and transcription inhibitor.

Other substances not included in the class of macrocyclic polyketides, such as, for example, the protein synthesis inhibitors tetracycline and chloramphenicol, the cell-wall synthesis inhibitors fosfomycin and vancomycin or the gyrase inhibitor novobiocin, do not cause a stimulation of luminescence of this kind (FIG. 3).

According to the models presented (FIGS. 1 and 2), the specificity of the biosensor system of the invention in the very preferred embodiment is determined by the MphR(A) repressor and by the binding thereof to the mph(A)-promoter region. Using methods such as DNA shuffling (Stemmer, 1994), a staggered-extension process (Zhao et al., 1998) or a new DNA shuffling method (Coco et al., 2001), it is possible to modulate the binding property of MphR(A). It is possible, for example, to alter the binding specificity or affinity of MphR(A) for particular macrocyclic polyketides. Furthermore, the DNA-binding and transcription-regulating properties of MphR(A) may be altered. It is also possible to alter the selectivity and sensitivity of the biosensor system by changing the nucleotide sequence of the DNA region to which MphR(A) binds. In addition, it is possible to influence the sensor properties by regulating the cellular availability of the MphR(A) protein or of other components of the biosensor system.

This readily leads to the possibility of altering the biological component of the biosensor system in such a way that it is possible to detect specifically substances which are not specifically indicated by the very preferred embodiment. Particularly interesting in this connection are biological biosensor components which can indicate, for example, non-ribosomally synthesized, cyclic peptides such as vancomycin or analogues. In principle, it is possible to optimize the selectivity and sensitivity of the biosensor system for a particular structure by using methods such as DNA shuffling (see above) or other mutative methods. It may be advantageous in such alterations of the biosensor properties, for example, to place the promoter of the mph(A) operon upstream of promoter-less genes of a selectable gene such as a resistance gene, for example a tetracycline-resistance gene. This may enable variants of the biosensor system to be filtered out positively on the basis of the stimulatable tetracycline resistance in the course of work to alter biosensor properties.

The biosensor system of the invention may also be used within the framework of combinatorial biosynthesis. Thus it is possible, for example, to use the biosensor system in order to indicate substances of a polyketide library as recently described (Tang et al., 2001). The detection method is independent of the anti-bacterial activity of the substances tested. In addition, it is also easily possible to form in a biosensor system of the present invention a further system which forms a polyketide library of this or a similar kind. This makes it possible to readily detect cells of microorganisms, which provide intracellularly biocombinatorially generated macrocyclic polyketides. If the reporter-gene product used is green fluorescent protein, such cells may be sorted using a fluorescence-activated cell sorter (FACS).

The biosensor system of the invention has a wide range of applications. It may be used, for example, in situ for detecting macrocyclic polyketides in the natural or anthropogenic, living or non-living environment. It may thus be used, for example, for detecting macrocyclic polyketides produced by concentrated or isolated microorganisms or other organisms (cf. Example 5). It may of course, also be used for detecting not only natural or genetically engineered but also synthetic macrocyclic polyketides. In a particular manner, it may be used in the laboratory or production environment, in a homogeneous assay within the framework of screening methods in reagent vessels or in the microtiter-plate format. It may also be applied in an agar-plate diffusion assay, in the course of a thin-layer chromatography (Shaw et al., 1997; Eberz et al., 1996), in the continuous format high-throughput screening (WO 99/30154) or in the sensor-layer format (DE-A 199 15 310).

The method of the invention may be carried out in a particular manner with application of an agar-plate diffusion assay. In the case of stimulation of luminescence, detection is possible already after approx. 2 hours of incubation. Usually, an overnight incubation (15 to 24 hours) will be advantageous.

It is also easily possible to spread the biosensor cells suspended in soft agar on a growth of test organisms, such as, for example, colonies, in order to detect, on the basis of a measurement such as, for example, measuring the stimulation of luminescence, an organism which produces a macrocyclic polyketide.

It is also possible, on the basis of the present invention, to develop a non-cellular screening assay for detecting macrocyclic polyketides by using subcellular components of the biosensor system of the invention, for example, particular proteins or co-factors.

The present invention also relates to the host cells forming the biosensor system of the invention and to corresponding DNA constructs.

The present invention furthermore relates to a kit which comprises the biosensor system of the invention.

### EXAMPLES

### Example 1

Protocol for Preparing a Luciferase Reporter Plasmid

Preparation of plasmid pEBZ511

The plasmid pEBZ511 was prepared by first obtaining the promoter of the mph(A operon and the DNA sequence to which the MphR protein binds, using PCR amplification. The template used was DNA of plasmid pTZ3509 (Noguchi et al., 2000). The primers used were the oligonucleotides mphf216 and mphr217 which served to amplify the desired DNA fragment with a terminal BamHI and EcoRI recognition sequence. The PCR mixture was first denatured at 95° C. for 3 minutes and then subjected to 30 cycles of denaturing at 95° C. for 60 seconds, annealing at −58° C. for 90 seconds and extension at 68° C. for 2 minutes. The PCR mixture contained 100 pmol of the two primers, 10 ng of pTZ3509, 0.5 mM of dNTPs, 1×Pfx buffer (Life Technologies), 1×PCR-enhancer solution (Life Technologies) and 5 U of Pfx polymerase (Life Technologies). The DNA amplicon obtained was isolated, cut with BamHI and EcoRI and cloned into the vector pUCD615 cut with BamHI and EcoRI (Rogowsky et al., 1987), using standard molecular-biological methods (Sambrook et. al., 1989).

| | |
|---|---|
| mphf216: | CGCGGATCCTGATGCGTGCACTACGCAAAGGCCAGG |
| mphr217: | CCGGAATTCAGTCAGCGGGCCATGGAGCTTGAGCCC |

### Example 2

Protocol for Preparing a Suicide Vector Carrying an mph(A) Operon

Preparation of the mph(A) Operon-Carrying Suicide Transposon Plasmid pEBZ512 and Transposition of the mph(A) Operon into E. coli SM101

Using PstI, the mph(A) operon was excised on a 4.1 kb DNA fragment from plasmid pTZ3509 (Noguchi et al., 2000) and cloned into the PstI cleavage site of the suicide transposon vector pBSL180 (Alexeyev and Shokolenko, 1995), into a transposable portion of the plasmid, using standard molecular-biological methods. The host used for transformation was E. coli CC118 Lambdapir (Herrero et al., 1990). This strain possesses the π protein which is required for replication of the plasmid. The prepared plasmid was referred to as pEBZ512. This plasmid was transferred using standard transformation methods into E. coli SM101 and transformants were selected after transposition of the mph(A) operon-carrying transposon. The mph(A) operon-carrying transposed piece of DNA which had been integrated into the genome by this procedure was referred to as EBZ512. The molecular-biological work (DNA isolations, restrictions, ligations and transformations) were carried out using standard methods (Sambrook et al., 1989).

### Example 3

Protocol for Preparing an mphR(A)-Encoding Plasmid

Preparation of the mphR(A)-Encoding Plasmid pEBZ514

The gene region of mphR(A) was amplified by means of PCR with the aid of primers MLV637 and MLV638 and pEBZ512-DNA. A ribosomal binding site and an SphI cleavage site were integrated into primer MLV637 and an SalI cleavage site was integrated into primer MLV638. The PCR reaction was carried out on an Eppendorf thermocycler, model Mastercycler Gradient. The PCR mixture was first denatured at 96° C. for 10 minutes and then subjected to 30 elongation cycles of denaturation at 96° C. for 60 seconds, annealing at 56° C. for 90 seconds and elongation at 72° C. for 40 seconds. The PCR mixture contained 100 pmol of the two primers, 10 ng of pEBZ512-DNA, 0.5 mM of dNTPs, 1×Pfx buffer (Life Technologies), 1×PCR-enhancer solution (Life Technologies) and 5 U of Pfx polymerase (Life Technologies). The DNA fragment produced was isolated, cut with restriction enzymes SphI and SalI and ligated into the vector pACYC184 (Chang und Cohen, 1978) which had likewise been cut with SphI and SalI. The mphR(A)-encoding region was thus located within the tetracycline-resistance gene, downstream of the corresponding resistance-gene promoter. The molecular-biological work (DNA isolations, restrictions, ligations and transformations) were carried out using standard methods (Sambrook et al., 1989).

MLV637:
TATATAGCATGCAAGAAGGAGATATACATATGCCCCGCCCCAAGCTCAA
GTCCGATGACGAGGTACTC
MLV638:
TTTATAGTCGACCAGGGACTCTGCACACCTCCGTTTACGCATGT

### Example 4

Luminescence Measurements in the Agar-Plate Diffusion Assay

The biosensor E. coli SM 101 (EBZ512, pEBZ511, pEBZ514) was grown aerobically in LB medium to the late-logarithmic growth phase, mixed into soft agar using standard micro-biological methods and spread out on LB-agar plates containing chloramphenicol (25 μg/ml) and kanamycin (30 μg/ml). After the soft agar had solidified, susceptibility assay discs which already contained the substance to be assayed or unloaded assay discs (Oxoid GmbH, Am Lippeglacis 4-8, 46483 Wesel, Germany) to which the substance to be assayed had been applied were placed on the agar. The agar-plate diffusion assay was incubated at 28° C. for 2 to 4 hours or 15 to 24 hours and luminescence was recorded using a Berthold Luminograph LB980 (FIGS. 3, 4 and 5). If luminescence is stimulated, it is detectable within a diffusion zone around the sample placed. If the sample assayed has, at the same time, an anti-bacterial activity against the sensor organism, which is noticeable as a zone of inhibition in the very preferred embodiment of the longer incubation, then the stimulation of luminescence is detectable outside this halo of inhibition in the sub-lethal concentration range.

### Example 5

Detection of a Macrolide-(erythromycin-) Producing Organism

Using the biosensor, it was also possible to distinguish a microorganism which is known as producer of a macrocyclic polyketide from another microorganism in situ.

For this purpose, the microbial biosensor cell was used in the agar-plate diffusion assay. Agar blocks were placed on the biosensor suspension embedded in soft agar. These agar blocks were covered either with Saccharopolyspora erythraea DSM 40517 described as erythromycin producer or with Streptomyces viridochromogenes DSM 40721 described as avilamycin producer. Another agar block contained no growth. A stimulation of luminescence was detectable only with the additionally applied erythromycin assay disc and the Sa. erythraea-covered agar block (FIG. 5).

### DESCRIPTION OF THE FIGURES

FIG. 1

Model of mph(A) operon regulation in E. coli (according to Noguchi et al., 2000, modified)

FIG. 2

Model of the very preferred embodiment of the biological component of the biosensor system. LuxC, D, A, B, E and LuxC, D, A, B and E, structural genes and, respectively, products of structural genes of Vibrio fischeri luciferase. mphR(A), MphR(A), see Noguchi et al., 2000.

FIG. 3

Agar-plate diffusion assay and luminescence measurements using the E. coli biosensor system. Illustration of the selectivity of the biological sensor component. The numbers given in the information on the test substances indicate the amounts of substance applied in μg.

FIG. 4

Agar-plate diffusion assay and luminescence measurements using the E. coli biosensor system. Exemplary illustration of the stimulation of luminescence of the biological sensor component as a function of time. The numbers given in the information on the test substances indicate the amounts of substance applied in μg.

FIG. 5

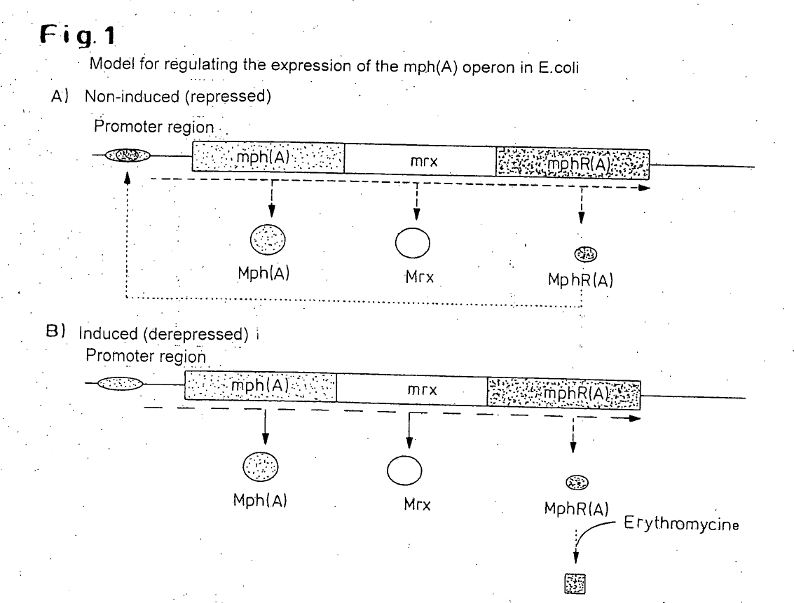

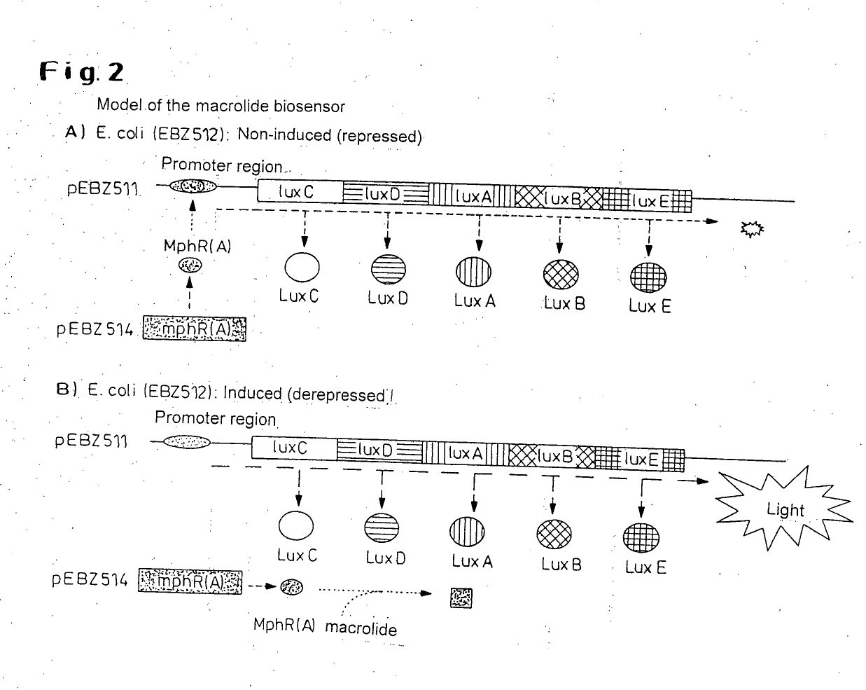

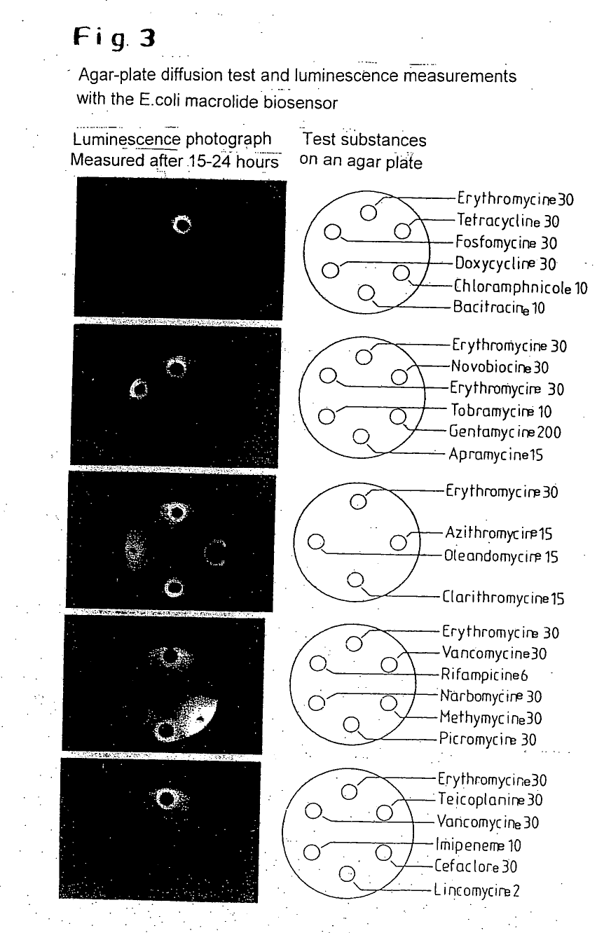

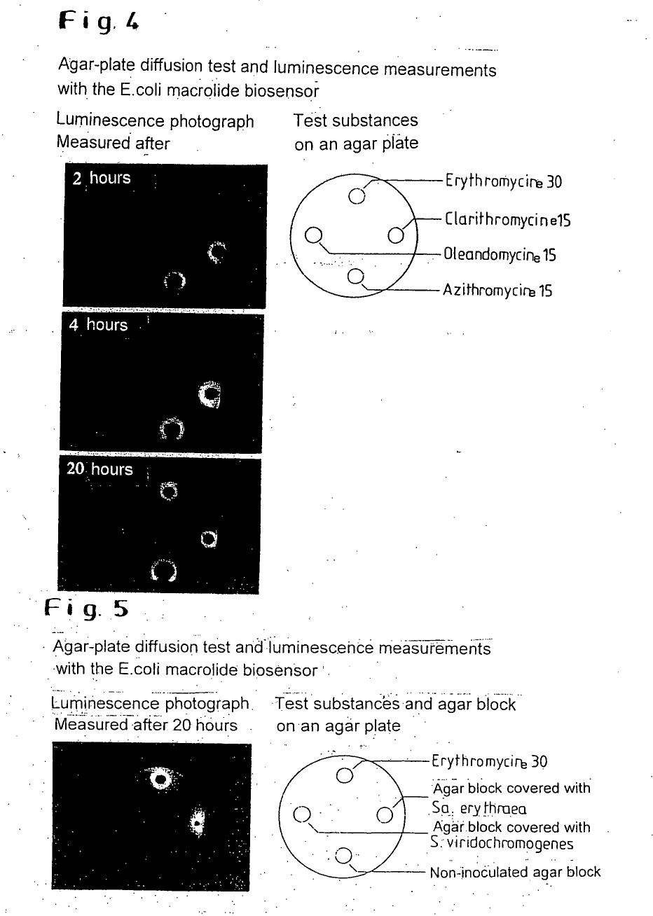

Agar-plate diffusion assay and luminescence measurements using the E. coli biosensor system. In situ stimulation of luminescence by an erythromycin-producing Saccharopolyspora erythraea culture. The number given in the information on the reference substance erythromycin indicates the amount of substance applied in μg.

### DEPOSITIONS

The following strains have been deposited with the Deutsche Sammlung von Mikroorganismen und Zellkulturen GmbH (DSMZ), Mascheroder Weg lb, D-38124 Brunswick, Germany, on Jun. 6, 2001 in agreement with the requirements of the Budapest Treaty:

| Name of strain | Deposition No. |
|---|---|
| E. coli SM101 (EBZ512, pEBZ511, pEBZ514) | DSM 14334 |
| E. coli SM101 (EBZ512) | DSM 14333 |

### REFERENCES

Alexeyev M., and Shokolenko I. N. (1995) Mini-Tn10 Transposon Derivatives for Insertion Mutagenesis and Gene Delivery into the Chromosome of Gram-negative Bacteria. Gene 160: 59-62.

Chang A. C. Y., and Cohen S. N. (1978) Construction and Characterization of Amplifiable Multicopy DNA Cloning Vehicles Derived from the P15A Cryptic Miniplasmid. J. Bacteriol. 134:1141-1156.

Coco W. M., Levinson W. E., Crist M. J., Hektor H. J., Darzins A., Pienkos P. T., Squires C. H., and Monticello D. J. (2001) DNA shuffling method for generating highly recombined genes and evolved enzymes. Nature Biotechnology 19: 354-359.

Daunert S., Barrett G., Feliciano J. S., Shetty R. S., Shrestha S., and Smith-Spencer W. (2000) Genetically Engineered Whole-Cell Sensing Sytems: Coupling Biological Recognition with Reporter Genes. Chem. Rev. 100: 2705-2738.

Eberz G., Rast H. G., Burger K, Kreiss W., Weisemann C. (1996) Bioactivity screening by chromatography-bioluminescence coupling. Chromatographia 43: 5-9.

Fralick J. A. (1996) Evidence that TolC is Required for Functioning of the Mar/AcrAB Efflux Pump in Escherichia coli. J. Bacteriol. 178: 5803-5805.

Herrero M., de Lorenzo V., and Timmis K. N. (1990) Transposon Vectors Containing Non-Antibiotic Resistance Selection Markers for Cloning and Stable Chromosomal Insertion of Foreign Genes in Gram-negative Bacteria. J. Bacteriol. 172: 6557-6567.

Kurittu J., Karp M., and Korpela M. (2000) Detection of Tetracyclines with Luminescent Bacterial Strains. Luminescence 15: 291-297.

Ludwig C., Ecker S., Schwindel K, Rast H. G., Stetter KO., and Eberz G. (1999) Construction of Highly Bioluminescent Nitrosomonas as a Probe for Nitrification Conditions. Arch. Microbiol. 172: 45-50.

Noguchi N., Takada K, Katayama J., Emura A., and Sasatsu M. (2000) Regulation of Transcription of mph(A) Gene for Macrolide 2′-Phosphotransferase I in Escherichia coli: Characterization of the Regulatory Gene mphR(A). J. Bacteriol. 182: 5052-5058.

Peitzsch N., Eberz G., and Nies D. H. (1998) Alcaligenes eutrophus as a Bacterial Chromate Sensor. Appl. Environ. Microbiol. 64: 453-458.

Pühler A., Regitz M., and Schmid R. D. (2000) Biochemie und Molekularbiologie. Römpp Lexikon, Georg Thieme Verlag Stuttgart—New York.

Rogowsky P. M., Close T. J., Chimera J. A., Shaw J. J., and Kado C. I. (1987) Regulation of the vir Genes of Agrobacterium tumefaciens Plasmid pTiC58. J. Bacteriol. 169: 5101-5112.

Sambrook J., Fritsch E. F., Maniatis T. (1989) Molecular Cloning: A Laboratory Manual. Cold Spring Harbor Laboratory Press.

Shaw P. D., Ping G., Daly S. L., Cha C., Cronan J. E., Rinehart K. L., and Farrand S. K (1997) Detecting and Characterzing N-acyl-homoserine Lactone Signal Molecules by Thin-layer-chromatography. Proc. Natl. Acad. Sci. USA 94: 6036-6041.

Stemmer W. P. C. (1994) DANN Shuffling by Random Fragmentation and Reassembly—in-vitro Recombination for Molecular Evolution. Proc. Nat. Acad. Sci. 91: 10747-10751.

Tang L., McDaniel R. (2001) Construction of Desosamine Containing Polyketide Libraries using a glycosyltransferase with broad substrate specificity. Chem. Biol. 8: 547-555.

Vuorio R., and Vaara M. (1992) The Lipid A Biosynthesis Mutations lpxA2 of Escherichia coli Results in Drastic Antibiotic Supersusceptibility. Antimicrob. Agents Chemother. 36: 826-829.

Zhao H. M., Giver L., Shao Z. X., Affholter J. A., and Arnold F. H. (1998) Molecular Evolution by Staggered Extension Process (StEP) in vitro Recombination. Nature Biotechnology 16: 258-261.

1. A method for identifying macrocyclic polyketides, comprising identifying macrocyclic polyketides or a mixture containing macrocyclic polyketides by subjecting said macrocyclic polyketide or said mixture to a biosensor system.

2. The method according to claim 1, wherein the macrocyclic polyketides are macrolides.

3. The method according to claim 1 wherein the biosensor system is a cellular reporter-gene assay system.

4. The method according to claim 3, wherein the cellular reporter-gene assay system comprises a reporter gene whose transcription is under the control of a promoter region which is regulated as a function of a macrocyclic polyketide.

5. The method according to claim 4, wherein the reporter gene used is a gene coding for chloramphenicol acetyltransferase, beta-galactosidase, luciferase or green fluorescent protein (GFP).

6. The method according to claim 5, wherein the reporter gene used is a gene coding for luciferase.

7. The method according to claim 4 wherein the promoter region is a promoter region of the E. coli mph(A) gene and the assay system additionally comprises the E. coli MphR(A) protein.

8. The method according to claim 7, wherein the MphR(A) protein is provided by overexpressing the mphR(A) gene.

9. The method according to claim 7 wherein the assay system additionally comprises the complete E. coli mph(A) operon or parts thereof.

10. The method according to claim 3 wherein the cells are bacteria.

11. The method according to claim 10, wherein the bacteria are E. coli.

12. The method according to claim 11, wherein said E. coli is SM101 (EBZ512, pEBZ511, pEBZ514), deposited under DSM 14334.

13. The method according to claim 11, wherein said E. coli is a strain having a mutation in the lpxA2 gene and/or the tolC gene.

14. A host cell comprising a reporter gene whose transcription is under the control of a promoter region which is regulated as a function of a macrocyclic polyketide.

15. The host cell according to claim 14, wherein the macrocyclic polyketide is a macrolide.

16. The host cell according to claim 14 wherein the reporter gene is a gene coding for chloramphenicol acetyltransferase, beta-galactosidase, luciferase or green fluorescent protein (GFP).

17. The host cell according to claim 16, wherein the reporter gene is a gene coding for luciferase.

18. The host cell according to claim 14 wherein the promoter region is the promoter region of the E. coli mph(A) gene and the host cell additionally comprises the E. coli MphR(A) protein.

19. The host cell according to claim 18, wherein the MphR(A) protein is provided by overexpressing the mphR(A) gene.

20. The host cell according to claim 18 wherein said host cell further comprises the complete E. coli mph(A) operon or parts thereof.

21. The host cell according to claim 14 wherein said host cell is a bacterial cell.

22. The host cell according to claim 21, wherein said host cell is E. coli.

23. The host cell according to claim 22, wherein said E. coli is SM 101 (EBZ512, pEBZ511, pEBZ514), deposited under DSM 14334.

24. The host cell according to claim 22, wherein said E. coli is a strain having a mutation in the lpxA2 gene and/or the tolC gene.

25. (Cancelled)

26. A DNA construct comprising a reporter gene having a promoter region which is regulated as a function of a macrocyclic polyketide.

27. The DNA construct according to claim 26, wherein the macrocyclic polyketide is a macrolide.

28. The DNA construct according to claim 26 wherein the reporter gene is a gene coding for chloramphenicol acetyltransferase, beta-galactosidase, luciferase or green fluorescent protein (GFP).

29. The DNA construct according to claim 28, wherein the reporter gene is a gene coding for luciferase.

30. The DNA construct according to claim 26 wherein the promoter region is the promoter region of the E. coli mph(A) gene.

31. (Cancelled)

32. A kit comprising a biosensor system as defined in claim 1 or a host cell according to claim 14.
